(19) United States
(12) Patent Application Publication
(10) Pub. No.: US 2026/0115236 A1
(43) Pub. Date: Apr. 30, 2026

(54) PROBIOTICS FOR MENTAL HEALTH

(71) Applicant: INTERNATIONAL N&H DENMARK APS, Kongens Lyngby (DK)

(72) Inventors: Elaine PATTERSON, Kotka (FI); Sixto Alvin IBARRA, Kotka (FI); Juliane HELLHAMMER, Trier (DE); Emilia ELLSIEPEN, Trier (DE)

(21) Appl. No.: 18/985,811

(22) Filed: Dec. 18, 2024

Related U.S. Application Data
(63) Continuation of application No. 17/609,113, now abandoned.

(30) Foreign Application Priority Data
May 6, 2019 (EP) 19172858.3

(51) Int. Cl. A61K 35/744; A61P 25/22

(52) U.S. Cl. CPC A61K 35/744; A61P 25/22

(57) ABSTRACT

The invention relates to a bacterium of the species Lacticaseibacillus paracasei (formerly known as Lactobacillus paracasei) and compositions comprising a bacterium of the species Lacticaseibacillus paracasei for use in alleviating the psychological and/or physiological response to psychosocial and/or psychological stress and promoting mental and overall well-being, in a mammal. The invention further relates to bacterium of the species Lacticaseibacillus paracasei and compositions comprising a bacterium of the species Lacticaseibacillus paracasei for use in preventing and/or treating a symptom affecting mental health and promoting mental and overall well-being, in a mammal. The invention further relates to methods and uses of said bacterium and/or compositions.

### FIELD OF THE INVENTION

This invention relates to bacteria of the species Lacticaseibacillus paracasei (formerly known as Lactobacillus paracasei) for use in alleviating the psychological and/or physiological response to psychosocial and/or psychological stress and promoting mental and overall well-being, in a mammal. This invention also relates to bacteria of the species Lacticaseibacillus paracasei for use in preventing and/or treating a symptom affecting mental health and promoting mental and overall well-being, in a mammal. This invention further relates to compositions, methods and uses of compositions comprising Lacticaseibacillus paracasei, including food products, dietary supplements, and pharmaceutically acceptable formulations/compositions.

### BACKGROUND

Mental health and overall well-being are related to emotional, psychological, physiological, physical and social well-being. Our mental health and overall well-being status are factors which can determine how we handle stress. A mental illness can be defined as a health condition that changes a person's thinking, feelings, or behaviour (or all three) and that causes the person distress and problems functioning in social, work or family activities. Mental illness encompasses a wide range of disorders related to anxiety, mood, psychosis, eating behaviour, impulse control and addiction, personality, sociability, dissociation, obsessive-compulsive and post-traumatic stress. Each illness alters a person's thoughts, feelings, and/or behaviours in distinct ways. Disorders such as Parkinson's disease, epilepsy and multiple sclerosis are brain disorders, but they are considered neurological diseases rather than mental illness. Interestingly, the lines between mental illness and neurological diseases, including memory disorders such as mild cognitive impairment, dementia and Alzheimer's disease, are not clearly defined and increasing evidence now suggests that mental illness is associated with changes in the brain's structure, chemistry and function which could underlie the development of neurological disorders. For example, the link between neurocognitive deficits and mood disorders is well established such that in major depression, cognitive impairment can mimic that observed in dementia (Rabins et al. Br J Psychiatry 1984; 144:488-92).

Stress is an individual process to deal with external and internal challenges that ranges from behavioural to molecular adaptations. The hypothalamic pituitary adrenal (HPA) axis and its release of stress hormones plays a major role in stress adaptation (D. H. Hellhammer et al., 2010; Mason, 1968; Selye, 1950). The HPA axis is a major part of the neuroendocrine system and presents a complex set of interactions between the hypothalamus, the pituitary and the adrenal glands. The HPA axis is involved in numerous processes such as e.g. energy balance, immune system and mood, and also controls reactions to stress (Bateman, Singh, Kral, & Solomon, 1989; Dallman & Hellhammer, 2011; D. H. Hellhammer et al., 2010; D. H. Hellhammer & Wade, 1993; Holsboer & Ising, 2010; Nieuwenhuizen & Rutters, 2008; Sapolsky, Romero, & Munck, 2000; Tsigos & Chrousos, 2002). In response to acute stressors, a hormonal cascade is activated: the hypothalamus secretes corticotrophin-releasing hormone (CRH), which triggers the release of adrenocorticotrophic hormone (ACTH) from the pituitary gland. ACTH then provokes the release of cortisol from the cortex of the suprarenal gland. Cortisol plays an important regulatory role in the carbohydrate, lipid and protein metabolism. Its catabolic effect causes the release of energy; additionally, cortisol has immunosuppressive and anti-inflammatory properties (Sapolsky et al., 2000).

Untreated chronic stress can result in serious health conditions such as anxiety, sleep problems, muscle pain, high blood pressure and a weakened immune system. Research shows that stress can contribute to the development of major illnesses, such as heart disease, depression and obesity. Symptoms of acute and chronic stress can manifest in the gastrointestinal tract, causing short- and long-term effects on the functions of the gastrointestinal tract, respectively. Exposure to stress results in alterations within the gut-brain axis, ultimately leading to the development of a broad array of gastrointestinal disorders including inflammatory bowel disease (IBD), irritable bowel syndrome (IBS) and other functional gastrointestinal diseases, food antigen-related adverse responses, peptic ulcers and gastroesophageal reflux disease (GERD). The major effects of stress on gut physiology include: 1) alterations in gastrointestinal motility; 2) increase in visceral perception; 3) changes in gastrointestinal secretion; 4) increase in intestinal permeability; 5) negative effects on regenerative capacity of gastrointestinal mucosa and mucosal blood flow; and 6) alteration in gut microbial composition (Konturek et al. J. Physiol Pharmacol. 2011; 62 (6): 591-9).

With respect to mental illness and associated neurocognitive decline and neurological disorders, there is now a clear emphasis on strategies to achieve positive mental and cognitive health for a full and healthy life. There is an increase in demand for nutritional therapies to achieve positive mental health, with no side effects. Current medication to treat symptoms of mental illnesses affecting mental health have many negative side effects such as nausea, increased appetite and weight gain, fatigue and gastrointestinal symptoms. Dietary supplements may represent an attractive means of achieving positive mental health and preventing symptoms of mental illness and related conditions from developing.

The gut-brain axis describes the bidirectional communication that exists between the brain and the gut and the microbiota-gut-brain axis supports the role of the gut microbiome in this communication system. As outlined above, mental illness and symptoms affecting mental health are comorbid with gastrointestinal disorders whereby emotional and routine daily stress can disrupt digestive function and vice versa. Increasing evidence indicates that the gut microbiota exerts a profound influence on brain physiology, psychological responses and ultimately behaviour (Dinan et al. J. Psychiatr Res. 2015; 63:1-9). Emerging evidence suggests that probiotics can influence central nervous system function and regulate mood, psychological symptoms such as anxiety and depression and stress-related changes in physiology, behaviour and brain function.

### OBJECT OF INVENTION

It is an object of the present invention to provide means for alleviating the psychological and/or physiological response to psychosocial and/or psychological stress and promoting mental and overall well-being, in a mammal. Furthermore, it is also the object of the present invention to provide means for preventing and/or treating a symptom affecting mental health and promoting mental and overall well-being, in a mammal. It is therefore an object of the invention to provide means by which an individual's mental and overall well-being can be promoted, maintained, and/or restored.

### SUMMARY OF THE INVENTION

The present invention is based on studies described herein which surprisingly demonstrate that Lacticaseibacillus paracasei can significantly alleviate the effects of psychosocial and/or psychological stress, prevent or treat symptoms affecting mental health and promote mental and overall well-being.

Accordingly, in one aspect, the invention provides a bacterium of the species Lacticaseibacillus paracasei for use in alleviating the psychological and/or physiological response to psychosocial and/or psychological stress and promoting mental and overall well-being, in a mammal.

In another aspect, the invention provides a bacterium of the species Lacticaseibacillus paracasei for use in preventing and/or treating a symptom affecting mental health and promoting mental and overall well-being, in a mammal.

In yet a further aspect, the invention provides a composition comprising a bacterium of the species Lacticaseibacillus paracasei for use in alleviating the psychological and/or physiological response to psychosocial and/or psychological stress and promoting mental and overall well-being, in a mammal.

In another aspect, the invention provides a composition comprising a bacterium of the species Lacticaseibacillus paracasei for use in preventing and/or treating a symptom affecting mental health and promoting mental and overall well-being, in a mammal.

In a further aspect, the invention provides a method for alleviating the psychological and/or physiological response to psychosocial and/or psychological stress and promoting mental and overall well-being in a mammal, comprising administering to the mammal a bacterium of the species Lacticaseibacillus paracasei.

In yet a further aspect, the invention provides a method for preventing and/or treating a symptom affecting mental health and promoting mental and overall well-being in a mammal, comprising administering to the mammal a bacterium of the species Lacticaseibacillus paracasei.

In yet a further aspect, the invention provides a use of a bacterium of the species Lacticaseibacillus paracasei for alleviating the psychological and/or physiological response to psychosocial and/or psychological stress and promoting mental and overall well-being, in a mammal.

In another aspect, the invention provides a use of bacterium of the species Lacticaseibacillus paracasei for preventing and/or treating a symptom affecting mental health and promoting mental and overall well-being, in a mammal.

### DESCRIPTION OF DRAWINGS

FIG. 1. Heart rate (beats per minute) in response to the Trier Social Stress Test (mean values for individual time windows±standard error of the mean) following 5 weeks of daily intervention with 1.75×10¹⁰ colony forming units of Lacticaseibacillus paracasei Lpc-37 in the active group and compared with the placebo group.

FIG. 2. Visual Analogue Scale-exhaustion (%) in response to the Trier Social Stress Test (mean values in individual time windows±standard error of the mean) following 5 weeks of daily intervention with 1.75×10¹⁰ colony forming units of Lacticaseibacillus paracasei Lpc-37 in the active group and compared with the placebo group.

FIG. 3. Systolic blood pressure (mmHg) in response to the Trier Social Stress Test (mean values in individual time windows±standard error of the mean) following 5 weeks of daily intervention with 1.75×10¹⁰ colony forming units of Lacticaseibacillus paracasei Lpc-37 in the active group and compared with the placebo group.

FIG. 4. Perceived stress (Perceived Stress Scale score) in response to the treatment (mean values in individual time windows±standard error of the mean) following 5 weeks of daily intervention with 1.75×10¹⁰ colony forming units of Lacticaseibacillus paracasei Lpc-37 in the active group and compared with the placebo group.

FIG. 5. Cortisol normalization at 8 pm (cortisol frequencies; number of people with normal 8 pm cortisol response) in response to the treatment following 5 weeks of daily intervention with 1.75×10¹⁰ colony forming units of Lacticaseibacillus paracasei Lpc-37 in the active group and compared with the placebo group.

FIG. 6. Diastolic blood pressure (mmHg) in response to the treatment following 5 weeks of daily intervention with 1.75×10¹⁰ colony forming units of Lacticaseibacillus paracasei Lpc-37 in the active group and compared with the placebo group.

FIG. 7. Sleep-related recovery throughout the study period (day 2-51) following 2 weeks of run-in period and 5 weeks of daily intervention with 1.75×10¹⁰ colony forming units of Lacticaseibacillus paracasei Lpc-37 in the active group and compared with the placebo group (mean values for individual time windows±standard error of the mean).

FIG. 8. Perceived health status throughout the study period (day 2-51) following 2 weeks of run-in period and 5 weeks of daily intervention with 1.75×10¹⁰ colony forming units of Lacticaseibacillus paracasei Lpc-37 in the active group and compared with the placebo group (mean values for individual time windows±standard error of the mean).

FIG. 9. Perceived productivity throughout the study period (day 2-51) following 2 weeks of run-in period and 5 weeks of daily intervention with 1.75×10¹⁰ colony forming units of Lacticaseibacillus paracasei Lpc-37 in the active group and compared with the placebo group (mean values for individual time windows±standard error of the mean).

### DETAILED DESCRIPTION OF INVENTION

### Bacteria

The bacteria used in aspects of the invention are bacteria of the species Lacticaseibacillus paracasei. In an aspect, the Lacticaseibacillus paracasei is strain Lpc-37 (formerly known as Lactobacillus paracasei Lpc-37), also known as DGCC4981 or Lbc81. Strain Lpc-37 is registered at the ATCC under deposit number PTA 4798 and at the DSMZ (Leibniz-Institut DSMZ-Deutsche Sammlung von Mikroorganismen und Zellkulturen GmbH, Inhoffenstr. 7B D-38124) under deposit number DSM32661, on 5 Oct. 2017.

The conditions for cultivation of strain Lpc-37 are as follows: pH before sterilisation: 6,40; Sterilisation 15 min at 121° C.; pH after sterilisation: 6,20; Oxygen relationship: microaerophilic; incubation temperature: 37° C.; incubation time: 18 h; short term storage at: −18° C.; interval of transfer: 5 years.

The conditions for long term storage of strain Lpc-37 are as follows: propagate in MRS broth 17 to 18 h at 37° C. until visible growth; dilute 1:1 with fresh medium, add 10% glycerol; freeze and store below −80° C., preferably in liquid nitrogen.

In a first aspect, the present invention provides a bacterium of the species Lacticaseibacillus paracasei for use in alleviating the psychological and/or physiological response to psychosocial and/or psychological stress and promoting mental and overall well-being, in a mammal.

In another aspect, the present invention provides a bacterium of the species Lacticaseibacillus paracasei for use in preventing and/or treating a symptom affecting mental health and promoting mental and overall well-being, in a mammal.

In one aspect, the psychological response to psychosocial and/or psychological stress is perceived exhaustion.

In another aspect, the physiological response to psychosocial and/or psychological stress is increased heart rate, and/or salivary cortisol production, and/or blood pressure.

In a further aspect, the symptom affecting mental health is increased salivary cortisol production, and/or blood pressure, and/or perceived stress, and/or perceived anxiety, and/or sleep disruptions.

In a further aspect, the symptom affecting mental health may contribute to a neuropsychiatric condition.

Neuropsychiatric condition includes degenerative diseases, such as dementia, Parkinson's disease, Alzheimer's disease; mood disorders such as depression; neurotic disorders such as anxiety disorder and sleep disorders such as sleep apnea, narcolepsy, insomnia and parasomnia.

According to the present invention, the mental and overall well-being is promoted by improvements to perceived health and/or perceived productivity.

According to one aspect of the present invention, the bacterium of the species Lacticaseibacillus paracasei is a probiotic of the species Lacticaseibacillus paracasei or a mixture thereof.

In yet a further aspect, the bacterium of the species Lacticaseibacillus paracasei is strain Lpc-37, registered at the DSMZ under deposit number DSM32661, on 5 Oct. 2017.

The Lacticaseibacillus paracasei may be used in combination with one or more other bacterial species which have the ability to exert positive health benefits on the host to which they are administered.

The Lacticaseibacillus paracasei may be used in any form (for example viable, dormant, inactivated or dead bacteria) provided that the bacterium remains capable of exerting the effects described herein. Preferably, the Lacticaseibacillus paracasei used in aspects of the invention is viable.

Preferably, the Lacticaseibacillus paracasei and, when used in aspects of the invention, other bacterial species, is suitable for human and/or animal consumption. A skilled person will be readily aware of specific strains of Lacticaseibacillus paracasei and other bacterial strains which are used in the food and/or agricultural industries and which are generally considered suitable for human and/or animal consumption.

Optionally, the Lacticaseibacillus paracasei and, when used in aspects of the invention, other bacterial strains, are probiotic bacteria. The term “probiotic bacteria” is defined as covering any non-pathogenic bacteria which, when administered live in adequate amounts to a host, confers a health benefit on that host. For classification as a “probiotic”, the bacteria must survive passage through the upper part of the digestive tract of the host. They are non-pathogenic, non-toxic and exercise their beneficial effect on health on the one hand via ecological interactions with the resident flora in the digestive tract, and on the other hand via their ability to influence the host physiology and immune system in a positive manner. Probiotic bacteria, when administered to a host in sufficient numbers, have the ability to progress through the intestine, maintaining viability, exerting their primary effects in the lumen and/or the wall of the host's gastrointestinal tract. They then transiently form part of the resident flora and this colonisation (or transient colonisation) allows the probiotic bacteria to exercise a beneficial effect, such as the repression of potentially pathogenic micro-organisms present in the flora and interactions with the host in the intestine including the immune system.

### Compositions

The term “composition” is used in the broad sense to mean the way something is composed, i.e. its general makeup. In aspects of the invention, the compositions may consist essentially of a single strain of Lacticaseibacillus paracasei bacteria (e.g. ATCC PTA 4798/DSM 32661).

Alternatively, the compositions may comprise a Lacticaseibacillus paracasei strain together with other components, such as other bacterial strains, biological and chemical components, active ingredients, metabolites, nutrients, fibres, prebiotics, etc.

In one aspect, the present invention provides a composition comprising a bacterium of the species Lacticaseibacillus paracasei for use in alleviating the psychological and/or physiological response to psychosocial and/or psychological stress and promoting mental and overall well-being, in a mammal.

In another aspect, the present invention provides a composition comprising a bacterium of the species Lacticaseibacillus paracasei for use in preventing and/or treating a symptom affecting mental health and promoting mental and overall well-being, in a mammal.

The psychological response to psychosocial and/or psychological stress is perceived exhaustion.

In another aspect, the physiological response to psychosocial and/or psychological stress is increased heart rate, and/or salivary cortisol production, and/or blood pressure.

The symptom affecting mental health is increased salivary cortisol production, and/or blood pressure, and/or perceived stress, and/or perceived anxiety, and/or sleep disruptions.

In one aspect, the symptom affecting mental health may contribute to a neuropsychiatric condition. The neuropsychiatric condition includes degenerative diseases, such as dementia, Parkinson's disease, Alzheimer's disease; mood disorders such as depression; neurotic disorders such as anxiety disorder and sleep disorders such as sleep apnea, narcolepsy, insomnia and parasomnia.

In yet a further aspect, the mental and overall well-being is promoted by improvements to perceived health and/or perceived productivity.

In another aspect, the bacterium of the species Lacticaseibacillus paracasei is a probiotic of the species Lacticaseibacillus paracasei or a mixture thereof.

In one aspect of the present invention, the bacterium of the species Lacticaseibacillus paracasei is strain Lpc-37, registered at the DSMZ under deposit number DSM32661, on 5 Oct. 2017.

In an aspect of the present invention, the composition is in the form of a food product, a dietary supplement or a pharmaceutically acceptable composition.

According to one aspect of the present invention, the composition is a spray-dried or freeze-dried composition.

According to another aspect of the present invention, the composition comprises a cryoprotectant.

In yet a further aspect of the present invention, the bacterium of the species Lacticaseibacillus paracasei is present in the composition in an amount between 10⁶ and 10¹², e.g. between 10⁸ and 10¹² colony forming units (CFU) per dose, optionally 10¹⁰ CFU per dose.

While it is not a requirement that the compositions comprise any support, diluent or excipient, such a support, diluent or excipient may be added and used in a manner which is familiar to those skilled in the art. Examples of suitable excipients include, but are not limited to, microcrystalline cellulose, rice maltodextrin, silicone dioxide, and magnesium stearate. The compositions of the invention may also comprise cryoprotectant components (for example, glucose, sucrose, lactose, trehalose, sodium ascorbate and/or other suitable cryoprotectants).

The terms “composition” and “formulation” may be used interchangeably.

Compositions used in aspects of the invention may take the form of solid, liquid, solution or suspension preparations. Examples of solid preparations include, but are not limited to: tablets, pills, capsules, granules and powders which may be wettable, spray-dried or freeze dried/lyophilized. The compositions may contain flavouring or colouring agents. The compositions may be formulated for immediate-, delayed-, modified-, sustained-, pulsed- or controlled-release applications.

By way of example, if the compositions of the present invention are used in a tablet form, the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of other acceptable carriers for use in preparing compositions include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, hydroxymethylcellulose, polyvinylpyrrolidone, and the like.

For aqueous suspensions and/or elixirs, the composition of the present invention may be combined with various sweetening or flavouring agents, colouring matter or dyes, with emulsifying and/or suspending agents and with diluents such as water, propylene glycol and glycerin, and combinations thereof.

Specific non-limiting examples of compositions which can be used in aspects of the invention are set out below for illustrative purposes. These include, but are not limited to food products, functional foods, dietary supplements, pharmaceutical compositions and medicaments.

### Dietary Supplements

The compositions of the invention may take the form of dietary supplements or may themselves be used in combination with dietary supplements, also referred to herein as food supplements.

The term “dietary supplement” as used herein refers to a product intended for ingestion that contains a “dietary ingredient” intended to add nutritional value or health benefits to (supplement) the diet. A “dietary ingredient” may include (but is not limited to) one, or any combination, of the following substances: bacteria, a probiotic (e.g. probiotic bacteria), a vitamin, a mineral, a herb or other botanical, an amino acid, a dietary substance for use by people to supplement the diet by increasing the total dietary intake, a concentrate, metabolite, constituent, or extract.

Dietary supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the invention may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the invention may take the form of one of the following:

A fruit juice; a beverage comprising whey protein: a health or herbal tea, a cocoa drink, a milk drink, a lactic acid bacteria drink, a yoghurt and/or a drinking yoghurt, a cheese, an ice cream, a water ice, a dessert, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

Optionally, where the product is a food product, the bacterium Lacticaseibacillus paracasei should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment.

### Food Ingredients

Compositions of the present invention may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the invention may take the form of functional foods.

As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction but is also capable of delivering a therapeutic (or other beneficial) effect to the consumer. Nutraceuticals cross the traditional dividing lines between foods and medicine.

### Medical Foods

Compositions of the present invention may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

### Pharmaceutical Compositions

Compositions of the invention may be used as—or in the preparation of—pharmaceuticals. Here, the term “pharmaceutical” is used in a broad sense—and covers pharmaceuticals for humans as well as pharmaceuticals for animals (i.e. veterinary applications). In a preferred aspect, the pharmaceutical is for human use.

The pharmaceutical can be for therapeutic purposes-which may be curative, palliative or preventative in nature.

A pharmaceutical may be in the form of a compressed tablet, tablet, capsule, ointment, suppository or drinkable solution.

When used as—or in the preparation of—a pharmaceutical, the compositions of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the form of a liquid or as a solid-depending on the use and/or the mode of application and/or the mode of administration.

The Lacticaseibacillus paracasei used in the present invention may itself constitute a pharmaceutically active ingredient. In one embodiment, the Lacticaseibacillus paracasei constitutes the sole active component. Alternatively, the Lacticaseibacillus paracasei may be at least one of a number (i.e. two or more) of pharmaceutically active components.

### Medicaments

Compositions of the invention may take the form of medicaments.

The term “medicament” as used herein encompasses medicaments for both human and animal usage in human and veterinary medicine. In addition, the term “medicament” as used herein means any substance which provides a therapeutic, preventative and/or beneficial effect. The term “medicament” as used herein is not necessarily limited to substances which need Marketing Approval but may include substances which can be used in cosmetics, nutraceuticals, food (including feeds and beverages for example), probiotic cultures, and natural remedies. In addition, the term “medicament” as used herein encompasses a product designed for incorporation in animal feed, for example livestock feed and/or pet food.

### Dosage

The compositions of the present invention may comprise from 10⁶ to 10¹² CFU of Lacticaseibacillus paracasei bacteria per dose or per gram of composition, and more particularly from 10⁸ to 10¹² CFU of Lacticaseibacillus paracasei bacteria per dose or per gram of composition. Optionally the compositions comprise about 10¹⁰ CFU Lacticaseibacillus paracasei per dose or per gram of composition.

The Lacticaseibacillus paracasei may be administered at a dosage from about 10⁶ to about 10¹² CFU of bacteria per dose, preferably about 10⁸ to about 10¹² CFU of bacteria per dose. By the term “per dose” it is meant that this number of bacteria is provided to a subject either per day or per intake, preferably per day. For example, if the bacteria are to be administered in a food product, for example in a yoghurt, then the yoghurt may contain from about 10⁶ to 10¹² CFU of Lacticaseibacillus paracasei. Alternatively, however, this number of bacteria may be split into multiple administrations, each consisting of a smaller amount of microbial loading-so long as the overall amount of Lacticaseibacillus paracasei received by the subject in any specific time, for instance each 24-hour period, is from about 10⁶ to about 10¹² CFU of bacteria, optionally 10⁸ to about 10¹² CFU of bacteria.

In accordance with the present invention an effective amount of at least one strain of a Lacticaseibacillus paracasei may be at least 10⁶ CFU of bacteria/dose, optionally from about 10⁸ to about 10¹² CFU of bacteria/dose, e.g., about 10¹⁰ CFU of bacteria/dose.

In one embodiment, the Lacticaseibacillus paracasei (e.g. ATCC PTA-4798/DSM 32661), may be administered at a dosage from about 10⁶ to about 10¹² CFU of bacteria/day, optionally about 10⁸ to about 10¹² CFU of bacteria/day. Hence, the effective amount in this embodiment may be from about 10⁶ to about 10¹² CFU of bacteria/day, optionally about 10⁸ to about 10¹² CFU of bacteria/day.

### Effects/Subjects/Medical Indications

The bacteria and/or compositions of the present invention can be used for administration to a mammal, including for example livestock (including cattle, horses, pigs, and sheep), and humans. In some embodiments of the present invention, the mammal is a companion animal (including pets), such as a dog or a cat for instance. In preferred embodiments, the bacteria and compositions are for use in a human.

The bacteria and/or compositions of the present invention can be used for alleviating the psychological and/or physiological response to psychosocial and/or psychological stress and promoting mental and overall well-being.

Furthermore, the bacteria and/or compositions of the present invention can be used for preventing and/or treating a symptom affecting mental health and promoting mental and overall well-being.

The term “mental illness” can be defined as a health condition that changes a person's thinking, feelings, or behaviour (or all three) and that causes the person distress and problems functioning in social, work or family activities. Mental illness encompasses a wide range of disorders related to anxiety, mood, psychosis, sleep, eating behaviour, impulse control and addiction, personality, sociability, dissociation, obsessive-compulsive and post-traumatic stress. Each illness alters a person's thoughts, feelings, and/or behaviours in distinct ways. As used herein, mental illness also includes neurological disorders and conditions related to mental illness which may be a cause or symptom of a mental illness or be a condition that can increase the chance of one developing.

In contrast, mental health is a level of psychological well-being or an absence of mental illness.

Disorders associated with anxiety are categorised under “mental illness”. The term “anxiety disorder” refers to a specific mental illness that involves extreme fear or worry, and includes generalized anxiety disorder (GAD), panic disorder and panic attacks, agoraphobia, social anxiety disorder, selective mutism, separation anxiety, and specific phobias. Obsessive-compulsive disorder (OCD) and posttraumatic stress disorder (PTSD) are closely related to anxiety disorders, which some may experience at the same time as depression. GAD represents more than the normal level of anxiety individuals experience from day to day and is characterised by chronic worry and tension. Compositions of the invention can be used to treat and/or prevent recognised anxiety disorders as well as symptoms of anxiety more generally.

As used herein, mental illness also includes associated neurological disorders, including memory disorders, mild cognitive impairment, dementia and Alzheimer's disease. Cognition denotes a relatively high level of processing of specific information including thinking, memory, perception, motivation, skilled movements and language. Cognitive disorders are defined as those with “a significant impairment of cognition or memory that represents a marked deterioration from a previous level of function” (Guerrero, Anthony (2008). Problem-Based Behavioural Science of Medicine. New York: Springer. pp. 367-79). They can be categorised into three main areas: (1) Delirium, a disorder affecting situational awareness and processing of new information; (2) Dementia, a disorder which can erase all or parts of an individual's memory; and (3) Amnesia, a disorder in which the individual afflicted has trouble retaining long term memories.

In one aspect of the present invention, the psychological response to psychosocial and/or psychological stress is perceived exhaustion.

In another aspect of the present invention, the physiological response to psychosocial and/or psychological stress is increased heart rate, and/or salivary cortisol production and/or blood pressure.

Symptoms affecting mental health are increased salivary cortisol production, and/or blood pressure, and/or perceived stress, and/or perceived anxiety, and/or sleep disruptions.

The symptom affecting mental health may contribute to a neuropsychiatric condition or mental illness. The neuropsychiatric condition includes degenerative diseases, such as dementia, Parkinson's disease, Alzheimer's disease; mood disorders such as depression; neurotic disorders such as anxiety disorder and sleep disorders such as sleep apnea, narcolepsy, insomnia and parasomnia.

The mental and overall well-being is promoted by improvements to perceived health and/or perceived productivity.

Other symptoms affecting mental health include; feeling sad or down, confused thinking or reduced ability to concentrate, excessive fears or worries, or extreme feelings of guilt, extreme mood changes of highs and lows, withdrawal from friends and activities, detachment from reality, paranoia or hallucinations, inability to cope with daily problems or stress, trouble understanding and relating to situations and to people, alcohol or drug abuse, major changes in eating habits, sex drive changes, excessive anger, hostility or violence and suicidal thoughts.

Mental illness and symptoms affecting mental health, also encompass conditions affecting an individual's cognitive function. Such conditions may include or overlap with various cognitive disorders. Examples include, but are not limited to, agnosia, amnesia, dementia, Alzheimer's disease, Parkinson's disease, and chronic stress, which has been shown to negatively affect brain function.

Intense acute and chronic stress can negatively impact both physical and mental health, increasing risk of developing mental illness. For example, chronic stress has been correlated with the development of mood disorders, anxiety disorders and depression. The compositions of the invention can be used to prevent and/or treat a mental illness or symptoms affecting mental health, resulting from chronic or acute stress.

### METHODS, USES AND OTHER EMBODIMENTS OF THE INVENTION

In one aspect, the present invention provides a method for alleviating the psychological and/or physiological response to psychosocial and/or psychological stress and promoting mental and overall well-being in a mammal, comprising administering to the mammal a bacterium of the species Lacticaseibacillus paracasei.

In another aspect, the present invention provides a method for preventing and/or treating a symptom affecting mental health and promoting mental and overall well-being in a mammal, comprising administering to the mammal a bacterium of the species Lacticaseibacillus paracasei.

In yet a further aspect, the invention provides for the use of a bacterium of the species Lacticaseibacillus paracasei for alleviating the psychological and/or physiological response to psychosocial and/or psychological stress and promoting mental and overall well-being, in a mammal.

In a further aspect, the invention provides for the use of bacterium of the species Lacticaseibacillus paracasei for preventing and/or treating a symptom affecting mental health and promoting mental and overall well-being, in a mammal.

For the avoidance of doubt, the bacteria and any of the compositions described in the present invention can be utilised in the methods and use aspects of the invention. For example, further embodiments include, but are not limited to, those set out below:

Embodiment 1: A method for alleviating the psychological and/or physiological response to psychosocial and/or psychological stress and promoting mental and overall well-being in a mammal, comprising administering to the mammal a bacterium of the species Lacticaseibacillus paracasei.

Embodiment 2: A method for preventing and/or treating a symptom affecting mental health and promoting mental and overall well-being in a mammal, comprising administering to the mammal a bacterium of the species Lacticaseibacillus paracasei.

Embodiment 3: The method according to embodiment 1, wherein the psychological response to psychosocial and/or psychological stress is perceived exhaustion.

Embodiment 4: The method according to embodiment 1, wherein the physiological response to psychosocial and/or psychological stress is increased heart rate, and/or salivary cortisol production and/or blood pressure.

Embodiment 5: The method according to embodiment 2, wherein the symptom affecting mental health is increased salivary cortisol production, and/or blood pressure, and/or perceived stress, and/or perceived anxiety, and/or sleep disruptions.

Embodiment 6: The method according to embodiment 2, wherein the symptom affecting mental health may contribute to a neuropsychiatric condition.

Embodiment 7: The method according to embodiment 6, wherein the neuropsychiatric condition includes degenerative diseases, such as dementia, Parkinson's disease, Alzheimer's disease; mood disorders such as depression; neurotic disorders such as anxiety disorder and sleep disorders such as sleep apnea, narcolepsy, insomnia and parasomnia.

Embodiment 8: The method according to embodiments 1 or 2, wherein the mental and overall well-being is promoted by improvements to perceived health and/or perceived productivity.

Embodiment 9: The method according to embodiments 1 or 2, wherein the bacterium of the species Lacticaseibacillus paracasei is a probiotic of the species Lacticaseibacillus paracasei or a mixture thereof.

Embodiment 10: The method according to any one of the embodiments 1 or 9, wherein the Bacterium of the species Lacticaseibacillus paracasei is strain Lpc-37, registered at the DSMZ under deposit number DSM32661, on 5 Oct. 2017.

Embodiment 11: Use of a bacterium of the species Lacticaseibacillus paracasei for alleviating the psychological and/or physiological response to psychosocial and/or psychological stress and promoting mental and overall well-being, in a mammal.

Embodiment 12: Use of bacterium of the species Lacticaseibacillus paracasei for preventing and/or treating a symptom affecting mental health and promoting mental and overall well-being, in a mammal.

Embodiment 13: The use according to embodiment 11, wherein the psychological response to psychosocial and/or psychological stress is perceived exhaustion.

Embodiment 14: The use according to embodiment 11, wherein the physiological response to psychosocial and/or psychological stress is increased heart rate, and/or salivary cortisol production and/or blood pressure.

Embodiment 15: The use according to embodiment 12, wherein the symptom affecting mental health is increased salivary cortisol production, and/or blood pressure, and/or perceived stress, and/or perceived anxiety, and/or sleep disruptions.

Embodiment 16: The use according to embodiment 12, wherein the symptom affecting mental health may contribute to a neuropsychiatric condition.

Embodiment 17: The use according to embodiment 12, wherein the neuropsychiatric condition includes degenerative diseases, such as dementia, Parkinson's disease, Alzheimer's disease; mood disorders such as depression; neurotic disorders such as anxiety disorder and sleep disorders such as sleep apnea, narcolepsy, insomnia and parasomnia.

Embodiment 18: The use according to embodiments 1 or 2, wherein the mental and overall well-being is promoted by improvements to perceived health and/or perceived productivity.

Embodiment 19: The use according to embodiments 1 or 2, wherein the bacterium of the species Lacticaseibacillus paracasei is a probiotic of the species Lacticaseibacillus paracasei or a mixture thereof.

Embodiment 20: The use according to any one of the embodiments 1 or 19, wherein the bacterium of the species Lacticaseibacillus paracasei is strain Lpc-37, registered at the DSMZ under deposit number DSM32661, on 5 Oct. 2017.

### EXAMPLES

The following examples are provided to demonstrate and further illustrate specific embodiments and aspects of the present invention and are not to be construed as limiting the scope thereof.

### Study Rationale.

The purpose of this clinical trial was to determine whether a bacterium derived from the species Lacticaseibacillus paracasei (Lpc-37) could modulate stress experienced by healthy male and female participants exposed to the Trier Social Stress Test (TSST). This randomized, double-blind, placebo-controlled, parallel-groups clinical trial was statistically powered to detect a significant modulation of the increase in heart rate (HR), as the primary outcome, in response to the TSST (psychosocial and/or psychological stress). Self-reporting inventories related to stress, anxiety, subjective feelings and sleep were completed by participants to identify the potential impact of probiotic supplementation on other psychological and physiological outcomes. Designed as a proof-of-concept study, the results of this study serve as an indication that the chosen study design is suitable to discover stress-related effects of probiotics.

### Study Design

### Overall Study Design and Plan.

This study was a two-arm, double-blind, randomized, placebo-controlled clinical trial with the TSST as a challenge triggering perceived and physiological stress responses.

The study population included healthy male and female adults 18-45 years (inclusive). A total of 120 participants were recruited and randomized into one of the two treatment groups (active or placebo) after stratifying for chronic stress (below or above age-related median score of the chronic stress subscale of the Trier Inventory for Chronic Stress (TICS) questionnaire) and sex. Participants with a chronic stress score of ≤13 were stratified into the low chronic stress subgroups and participants with a chronic stress score of ≥14 were stratified into the high chronic stress subgroups.

Sixty (60) participants were assigned to the active group and received a capsule containing Lacticaseibacillus paracasei Lpc-37 at 1.75×10¹⁰ CFU each day for 5 weeks. Sixty (60) participants were assigned to the placebo group and received a capsule of the same form containing a matched placebo each day for 5 weeks.

The study was conducted in full accordance with the last Revisions from 2008 and 2013 of the Declaration of Helsinki, the European Medicines Agency Note for Guidance on Good Clinical Practice (CPMP/ICH/135/95-in operation 17.01.1997), also known as International Conference on Harmonization guidelines for Good Clinical Practice, and with laws and regulations for clinical research in Germany.

The study visits were defined as; Screening Visit (V1), Baseline Visit (V2), and post 5 weeks of intervention with either the active or placebo treatment, the Post-Treatment Visit (V3).

| | No. of visit | |
|---|---|---|
| Study day | at study site | Investigation steps |
| | V0 | Telephone Screening: |
| | | Interested persons received information via phone and a first check of |
| | | inclusion/exclusion criteria was performed. Eligible persons were |
| | | scheduled for a screening visit (V1) at the study site. |
| 1 | V1 | Screening Visit: |
| | | Interested persons were informed about the study procedure extensively. |
| | | Eligibility criteria were checked, and persons signed an informed consent. |
| | | The TICS questionnaire was filled in for stratification and participants |
| | | received a randomization number. Enrolled participants received a training |
| | | on how to enter their data into the daily online diary and on how to collect |
| | | saliva samples at home during the 2 days before V2. A saliva collection kit |
| | | was dispensed. |
| 2-15 | | Run-In: |
| | | During the two-week run-in period, randomized participants were not |
| | | permitted to consume products containing concentrated sources of |
| | | probiotics/prebiotics. Participants filled in the daily online diary each |
| | | morning. Saliva samples were collected during the 2 days before V2. |
| 16 | V2 | Baseline Visit: |
| | | Participants returned their saliva samples. Weight, heart rate (HR) and |
| | | blood pressure (BP) were measured. Baseline questionnaires (State-Trait |
| | | Anxiety Inventory (STAI-Trait, STAI-State), Visual Analogue Scale (VAS)- |
| | | stress, anxiety, insecurity and exhaustion, Beck Anxiety Inventory (BAI), |
| | | Depression Anxiety Stress Scale (DASS), Perceived Stress Scale (PSS)) |
| | | were assessed. The investigational product (IP) was dispensed and |
| | | product intake was explained. A second saliva collection kit was handed |
| | | over for post-treatment assessment during 2 days prior to V3. |
| 17-51 | | Treatment: |
| | | Participants took 1 capsule of the IP and filled in the online diary daily each |
| | | morning. Saliva samples were collected during the 2 days before V3. |
| 51 | V3 | Post-Treatment Visit: |
| | | After 5 weeks of IP intake participants returned to the study site. Saliva |
| | | samples were returned. Overall compliance with the study protocol was |
| | | checked and AEs were assessed. Weight, HR and BP were measured. |
| | | Post-treatment questionnaires (STAI-State, VAS stress, anxiety, insecurity |
| | | and exhaustion, BAI, DASS, PSS) were assessed. |
| | | The TSST was conducted. |
| | | Six saliva samples for the assessment of cortisol and salivary Alpha |
| | | Amylase (sAA) were collected before and after the TSST. A continuous HR |
| | | measurement took place before, during and after the TSST. BP was |
| | | assessed before and after the TSST. STAI-State anxiety was assessed |
| | | before and after the TSST, while VAS stress, anxiety, insecurity and |
| | | exhaustion were assessed before, during and after the TSST. |
| | | Completed participants received their compensation for participation in the |
| | | study. |
| 52-82 | | Adverse Events (AEs) still ongoing at V3 were followed-up by the study |
| | | manager up to 30 days after V3. |

### Selection of Study Population.

### Inclusion Criteria:

In order to participate in the study, the participants had to meet all of the following inclusion criteria:
Voluntary, written, informed consent to participate in the study
Male or female aged between 18-45 years (inclusive)
Body mass index (BMI) between 18.5-29.9 kg/m²
Medical examination at baseline indicates they are healthy in the opinion of the investigator
Ability of the participant (in the Principal Investigator's opinion) to comprehend the full nature and purpose of the study including possible risks and side effects
Agreement to comply with the protocol and study restrictions
Available for all study visits
Females of child-bearing potential required to provide a negative urine pregnancy test and to use contraceptives
Easy access to internet

### Exclusion Criteria:

Meeting any of the following exclusion criteria prevented participation in the study:
Self-reported diagnosis of one or more DSM-IV axis 1 (Diagnostic and Statistical Manual of Mental Disorders, 4th Edition, a manual published by the American Psychiatric Association that includes all currently recognized mental health disorders) disorder(s), including but not limited to current major depression, anxiety disorder, bipolar spectrum disorder or schizophrenia
Have a significant acute or chronic coexisting illness (cardiovascular, gastrointestinal (IBS, inflammatory bowel disease (IBD)), immunological, metabolic, neurodevelopmental or any condition which contraindicates, in the Investigator's judgement, entry to the study
Currently taking (from day of screening onwards) or have previously taken (last 4 weeks prior to screening) psychoactive medication (anxiolytics, sedatives, hypnotics, anti-psychotics, anti-depressants, anti-convulsants, centrally acting corticosteroids, opioid pain relievers)
Currently taking (from day of screening onwards) medication or dietary supplements that the Investigator believes would interfere with the objectives of the study, pose a safety risk or confound the interpretation of the study results (e.g. melatonin, omega-3 dietary supplements, non-steroidal anti-inflammatory drugs (NSAIDS), over-the-counter (OTC) sleep medication (not categorized as sedatives, hypnotics or anti-depressants), anti-coagulants, proton pump inhibitors, anti-histamines, pseudoephedrine, cortisone, beta-blockers)
Recent (within last 4 weeks prior to screening) or ongoing antibiotic therapy during the intervention period
Daily consumption of concentrated sources of probiotics and/or prebiotics within 2 weeks of screening and throughout the intervention period other than the provided study products (e.g., probiotic/prebiotic tablets, capsules, drops or powders)
Pregnant or lactating female, or pregnancy planned during intervention period
Not fluent in German
Have self-reported dyslexia
History of alcohol, drug, or medication abuse
Self-declared illicit drug users (including cannabis and cocaine) for 3 weeks prior to screening and during the intervention period
Contraindication to any substance in the investigational products (IP) s
Hypertension (systolic ≥140 mmHg, diastolic ≥90 mmHg)
Known hyper- or hypothyroidism unless treated and under control (stable for more than 3 months)
Persons having previously participated in the TSST
Smoking >5 cigarettes/day
Employee of the sponsor or CRO
Participation in another study with any investigational product within 60 days of screening and during the intervention period
Investigator believes that the participant may be uncooperative and/or noncompliant and should therefore not participate in the study
Participant under administrative or legal supervision

### Methods of Assigning Participants to Treatment Groups.

At the first contact during the telephone interview, all individuals received a consecutive screening number starting from S001. Informed consent was obtained before enrolment at V1 after which participants were identified with an individual randomization number. Enrolled individuals were stratified based on sex and their current stress level measured by the TICS. Participants with a chronic stress score of ≤13 were stratified into the low chronic stress subgroups, participants with a chronic stress score of ≥14 were stratified into the high chronic stress subgroups. The stratification was based on the median score of the age-related population. (Schulz P, Schlotz W, Becker P. Trierer Inventar zum chronischen Stress: TICS: Hogrefe, 2004; Schulz P, Schlotz W. Trierer Inventar zur Erfassung von chronischem Sre (TICS): Skalenkonstruktion, teststatistische Überprüfung und Validierung der Skala Arbeitsüberlastung. Diagnostica, 1999). Participants were sequentially enrolled by the study manager and assigned to the next free randomization number. Participants that dropped out of the study following randomization were not replaced, as per the study protocol.

### Investigational Products.

Out of a total study population of N=120, N=60 participants were randomly assigned to the active group. One participant who was assigned to the active group at V1 dropped out during the run-in period and therefore N=59 participants began the intervention and received a capsule containing Lacticaseibacillus paracasei Lpc-37 at 1.75×10¹⁰ CFU, microcrystalline cellulose, magnesium stearate and silicon dioxide each day for 5 weeks.

The other N=60 participants were randomly assigned to the placebo group. One participant who was assigned to the placebo group at V1 dropped out during the run-in period and therefore N=59 participants began the intervention and received a capsule of the same form containing microcrystalline cellulose, magnesium stearate and silicon dioxide each day for 5 weeks (matched placebo).

Active and placebo IPs were identical in appearance and taste. The IPs were consumed once per day for 5 weeks. The IP was provided to each participant at V2, with a 5-week supply plus some extra capsules in case of capsule misplacement. Participants were instructed to take one capsule of the IP every morning at least 30 minutes before breakfast, or their first meal of the day, with a glass of plain (not sparkling) water. Participants were surveyed about their overall compliance with the study protocol every morning in the online diary and the returned remaining capsules were counted by the study personnel at V3 to calculate compliance of IP intake.

### Blinding Procedure.

The unblinded randomization list was sent by 4Pharma Oy Ltd. to DuPont Nutrition and Health, Danisco Madison Center for packaging and labelling of IPs. DuPont Nutrition and Health Madison Center delivered the IPs together with individual blinded envelopes to daacro GmbH & Co. KG. 4Pharma provided the first key treatment code to the statistician at daacro GmbH & Co. KG after the blind data review had been completed, following instruction from DuPont Nutrition and Health. The second key treatment code was provided to the statistician at daacro GmbH & Co. KG after data analysis and the completion of the Clinical Study Report (CSR).

### Self-Report Scales.

At all visits, participants completed several self-report scales. Instructions on how to complete each self-report scale were explained by the study team during the study visits.

Trier Inventory for Chronic Stress (TICS; Schulz, Schlotz, & Becker, 2004).

The questionnaire consisted of 57 items. Stress chronicity was measured by the frequency of stressful events perceived retrospectively within the last 3 months. Answers were given on a five-point rating scale, where 0 resembles “never” and 4 “very often”. For analysis, the questionnaire items were assigned to 10 scales: Work overload, Social overload, Pressure to succeed, Work dissatisfaction, Excessive demands at work, Lack of social recognition, Social stress, Social isolation and Chronic worrying; the last scale presents a Screening scale for chronic stress.

It is not recommended to accumulate a total score of all items. To assess chronic stress, the Screening scale of chronic stress was provided which includes scores on twelve items from five scales (Chronic worrying, Work and Social overload, Excessive demands and Lack of acceptation). Scores were obtained by summing up the values of the scale specific questionnaire items. The respective score ranges are as follows: Work overload 0-32, Social overload 0-24, Pressure to succeed 0-36, Dissatisfaction with work 0-32, Excessive demands 0-24, Lack of acceptation 0-16, Social tension 0-24, Social isolation 0-24, Chronic worrying 0-16 and Screening scale of chronic stress 0-48. Scores of ≥24 on the Screening scale of chronic stress meet the criteria of being chronically stressed (above average of the age-related population; n=149).

The TICS was assessed at the Screening Visit (V1). Participants with a chronic stress score of ≤13 were stratified into the low chronic stress subgroups, participants with a chronic stress score of ≥14 were stratified into the high chronic stress subgroups. The stratification is based on the median score of the age-related population.

### State-Trait-Anxiety Inventory (STAI; Laux, 1981)

The STAI comprises two scales with 20 items each and assess (1) anxiety as a personality trait (STAI-X2) and (2) anxiety as a temporary emotional state (STAI-X1). Momentary state anxiety is characterized by tension, solicitude, nervousness, uneasiness and fear of future situations all of which are associated with an elevated Autonomic Nervous System (ANS). On the other hand, trait anxiety characteristics represent a stable tendency of a person and are therefore independent of time. State anxiety is correlated with trait anxiety, i.e. participants with an anxious personality perceive momentary situations as more threatening than participants with less trait anxiety.

Answers are given on a four-point rating scale ranging from 1=“not at all” to 4=“very true” for STAI-state (X1) and from 1=“almost never” to 4=“almost always” for STAI-trait (X2). Some STAI questions relate to the absence of anxiety and are reversely scored. For analysis of each STAI-scale, items are combined in one scale, which then informs about state or trait anxiety. The score range is 20-80; higher scores indicate more anxiety.

The STAI-trait was completed for baseline measurements at V2. In addition, state anxiety was assessed at V2 and at V3, as well as before and after the TSST (V3) using the STAI-state.

### Visual Analog Scale (VAS; Bond & Lader, 1974)

The VAS is a 10 cm bipolar visual scale ranging from “not at all” to “highly”. For this study, stress perception, anxiety, insecurity and exhaustion were assessed at five time points: at V2 and at V3, as well as before, during and immediately after the TSST (V3). The participant indicated his/her actual perception by placing a mark on a line.

VAS scores were obtained by using a ruler and measuring the position of the participant's mark with millimeter precision. To control for possible variations due to printing, the total length of the line was also measured and percentage scores for each participant were computed. Percentage scores range from 0-100. The VAS is a suitable and useful tool in order to measure perceived stress in reaction to the TSST (J. Hellhammer & Schubert, 2012).

### Depression Anxiety Stress Scales (DASS; S. Lovibond & Lovibond, 1995)

Participants completed the DASS at V2 and V3. The DASS gives information about negative emotional states of depression, anxiety and stress during the past week. The questionnaire includes three scales (depression, anxiety and stress) of which each scale includes 14 items that are divided into subscales of 2-5 items of similar content.

With the depression scale the following features are determined: dysphoria, hopelessness, devaluation of life, self-deprecation, and lack of interest/involvement, anhedonia, and inertia. The anxiety scale measures autonomic arousal, skeletal muscle effects, situational anxiety, and subjective experience of anxious effect.

The questionnaire has internal consistency and good reliability in healthy individuals and patient populations (Antony, Bieling, Cox, Enns, & Swinson, 1998; Brown, Chorpita, Korotitsch, & Barlow, 1997; Crawford & Henry, 2003; P. F. Lovibond, 1998).

### Perceived Stress Scale (PSS; Cohen, Kamarck, & Mermelstein, 1994)

Participants completed the PSS at V2 and V3. The PSS is a widely used psychological instrument for measuring stress perception. It assesses how unpredictable, uncontrollable and overloaded participants perceived their lives to have been within the last month. The PSS comprises 14 items that are answered on a five-point rating scale ranging from 0=“never” to 4=“very often”.

### Beck Anxiety Inventory (BAI; Margraf, Beck, & Ehlers, 2007)

Participants completed the BAI at V2 and V3. The BAI is a self-rating scale designed to measure anxiety in grown-ups and youths. It comprises 21 sentences describing feelings that can occur when being anxious. These sentences are rated on a four-point rating scale ranging from 0=“not at all” to 3=“severely”, considering the last 7 days.

### Participant Online Diary

Starting on day 2, participants filled out an online diary, daily. They were asked about health problems/adverse events, concomitant medications, sleep quality, health, well-being and mood.

Participants received an individual access to the online diary and were asked to complete the diary every day between 3 am and 12 pm. If entries were not performed in a timely manner, the study team received an e-mail notification. The respective participants were then contacted by a member of the study team and asked to submit the information as soon as possible. The study team could also add the information to the diary if the participant preferred to give the information over the phone.

### Trier Social Stress Test (TSST; Kirschbaum, Pirke, & Hellhammer, 1993).

In a meta-analysis of laboratory studies, Dickerson and Kemeny (2004) confirmed that the TSST is the most standardized and most effective protocol for inducing an endocrine reaction to psychosocial stress experimentally. During the TSST, the uncontrollable performance task associated with a social-evaluative threat and forced failure triggers the release of cortisol. The TSST has been reviewed in detail by Hellhammer, Kudielka and colleagues (D. H. Hellhammer, Stone, Hellhammer, & Broderick, 2010; B. Kudielka, Wüst, Kirschbaum, & Hellhammer, 2007; B. M. Kudielka, Hellhammer, Kirschbaum, Harmon-Jones, & Winkielman, 2007; B. M. Kudielka & Wüst, 2010).

The TSST lasted 15 min. and included an introduction by the study manager or a member of the study team, a preparation phase, an interview for a job in a new company and a mental arithmetic task.

Introduction (2 min.): The study manager or a member of the study team led the participant into the TSST room and introduced the TSST setting. Then the participant was asked to imagine applying for a job and was invited for an interview. The participant had 3 min. to prepare for this interview in the presence of two members of the committee board. The participant was informed that she/he was video, and voice recorded during the interview. In addition, she/he learned that both members of the committee were trained in behavioral observation and document her/his behavior during the interview as well as the manner and content of her/his speech. Finally, the study manager or a member of the study team asked the participant whether she/he had any questions regarding the protocol. Questions were answered directly. Thereafter, the participant was asked to start preparing the speech. The study manager or a member of the study team left the TSST room.

Preparation time (3 min.): The participant was allowed to take notes in preparation for the job interview.

Interview (5 min.): The participant was asked by a member of the committee board to step forward to the microphone and to present an interview for a job in a new company. Following the interview, the participant was asked to complete the VAS.

Mental arithmetic task (5 min.): The participant was asked to stepwise subtract 17 from 2023 as quickly and correctly as possible. After 15 minutes total, the participant was led back to his/her room by a member of the study team.

Times were documented by members of the committee board during the TSST.

The TSST was performed on V3.

### Physiological Measures.

### The Autonomic Nervous System (ANS)-Heart Rate (HR)

The ANS affects perspiration, heart rate, digestion, arousal, respiration, diameter of pupils, salivation, and regulates and adapts the visceral organs to strains and challenges of psychological as well as physiological influences and changes. Therewith, the ANS prepares the body for stress, but also for returning to relaxation phases afterwards. The ANS is divided into two antagonistic parts that perform these tasks. The sympathetic nervous system activates the organism and sets it up for stressors and strains. In contrast, relaxation is part of the parasympathetic nervous system. Necessarily, a high level of regulation of the ANS and a balanced relation of its subsystems are needed for an effective functioning. Pathological impacts on health, physical condition and ability of coping with psychological and physiological stress can be caused by disruptions of the system.

For determination of physiological, stress-induced changes of the ANS, HR was recorded for 55 minutes including time periods before, during and after the TSST on V3.

HR was recorded using a Polar watch device (M400, Polar Electro GmbH, Büttelborn, Germany). The Polar watch recorded data every second throughout the 55 min. test period. Non-personalized data was analyzed using https://flow.polar.com and was exported as csv-files.

Mean values were calculated for 7 different time windows:
10 min. sitting before the TSST
10 min. standing before the TSST
5 min. introduction to the TSST and preparation time
5 min. interview.
5 min. mental arithmetic task.
10 min. standing after the TSST.
10 min. sitting after the TSST

### Saliva Samples

Participants were asked to collect saliva samples for analysis of cortisol and sAA.

For determination of the Cortisol Awakening Response (CAR):

Saliva collection took place on two consecutive working days before V2 and on two consecutive working days before V3. Saliva samples were collected four times in the first hour after waking (awakening, 30, 45 and 60 min. after awakening), as well as at 8 pm that evening. This resulted in 20 saliva samples per participant, overall throughout the study.

For determination of stress induced changes of cortisol and sAA:

Saliva samples were collected before and after inducing acute stress by performing the TSST at V3. The first sample was collected 1 min. prior to the TSST to assess the baseline level. The remaining samples were collected at +1 min., +10 min., +20 min., +30 min. and at +45 min. with respect to end of the TSST.

For CAR- and TSST-related saliva collection, Salivette® Cortisol, code blue (Sarstedt, Nuembrecht, Germany) were used. CAR sampling took place at home and therefore participants received a detailed instruction on method and storage. Sampling itself was done by chewing a synthetic swab that collected the saliva and which acted as an insert to a collection tube for 1 min at each time point. Participants were asked to store the samples in the freezer or refrigerator at home. For return of the samples, participants were asked to bring them to the study site at their next scheduled visit (V2 and V3) without any further cooling during the transport needed.

Until further analysis, saliva samples were stored at the study site at −20° C. Saliva samples were analyzed at daacro's Saliva Lab Trier.

### Cortisol

The human brain uses about 80% of overall available energy reserves. Its energy balance is sustained by the release of the hormone cortisol, which mobilizes the energy supplier glucose. During stress, cortisol levels increase accordingly. Chronic stress can lead to health problems if this hormone is released in excessively low or high quantities. Careful analysis of cortisol release is therefore necessary in stress diagnosis.

Cortisol belongs to the hormonal group of glucocorticoids and is produced by the zona fasciculata in the adrenal gland. Cortisol has a diurnal pattern that is characterized by a rapid increase following morning awakening with a peak 30-45 min after awakening (Stalder et al., 2016). Thereafter cortisol levels steadily decline throughout the day. The CAR is a physiological reaction to awakening. The CAR is influenced by a variety of factors such as anticipation of the upcoming day (Fries, Dettenborn, & Kirschbaum, 2009), gender, health status and stress-related parameters, i.e. low socioeconomic status (Wright & Steptoe, 2005), work overload and chronic worrying (Schlotz, Hellhammer, Schulz, & Stone, 2004), social stress and the lack of social recognition (Wust, Federenko, Hellhammer, & Kirschbaum, 2000). Since the CAR is influenced by trait as well as by state factors, saliva samples of at least two days are needed to obtain reliable trait measures (J. Hellhammer et al., 2007).

Salivary cortisol levels were determined employing a high sensitivity salivary cortisol enzyme immunoassay kit (Salimetrics, LLC, USA).

### Alpha-Amylase

SAA is an enzyme that degrades starch into maltose and dextrin. It is secreted from the salivary glands in response to adrenergic stimulation. Rohleder, Nater, Wolf, Ehlert, and Kirschbaum (2004) describe a diurnal rhythm for sAA, with lowest levels in the morning and highest in the late afternoon.

Unlike cortisol, which is a biomarker of the stress response by the HPA system, alpha-amylase is considered to be a biomarker of the stress response by the sympatho-adreno-medullary system (Soo-Quee Koh & Choon-Huat Koh, 2007). Several authors have found correlations between sAA and stress-related activities, e.g. anxiety from arithmetic stress, TSST, adrenergic blockade with beta-blockers, physical exercise, examination stress and extreme temperature stress (Chatterton, Vogelsong, Lu, Ellman, & Hudgens, 1996; Nater et al., 2006; Noto, Sato, Kudo, Kurata, & Hirota, 2005; Takai et al., 2004; van Stegeren, Rohleder, Everaerd, & Wolf, 2006).

SAA levels were determined using a kinetic enzyme assay kit (Salimetrics, LLC, USA).

### Vital Parameters

At the Screening Visit (V1), blood pressure (BP) and pulse were obtained. BP (systolic and diastolic) and pulse were assessed by using an automated BP measurement device (OMRON M10-IT, OMRON Medizintechnik Handelsgesellschaft mbH, Gottlieb-Daimler-Str. 10, 68165 Mannheim). If BP measures were above the normal range (systolic ≥140, diastolic ≥90), a second measurement was taken. A systolic BP ≥140, and/or a diastolic BP ≥90 in both measurements led to participant exclusion from the study according to the exclusion criteria as per the protocol.

At V2 and V3, body mass index (BMI), BP and pulse were obtained following participant arrival at site. In addition, at V3, BP and pulse were measured before and after the TSST and HR continuously for 55 min before, during and after the TSST.

### Statistical Methods and Analytical Plans.

Statistical analyses were conducted using R Version 3.5.2 (R Core Team, 2018). R packages used include MuMIn, Hmisc, Ime4, car, influence.ME, Rmisc, ImerTest and coin.

Efficacy analyses were planned as linear mixed model (lmm) analyses and repeated measures analyses of variance (RM ANOVA) or alternatively as t-tests, Friedman analyses of variance, Wilcoxon Signed-Rank test, Kruskal Wallis test, and Mann Whitney U tests in case of violation of model assumptions. Two-sided hypothesis testing (α=0.05) was performed.

Mixed models were built up gradually, first testing how many time polynomials should be included, then testing possible covariates, and lastly adding the effect of treatment group and time×group interaction terms (e.g. time1×group, time2×group). Models were build including time and intercept as random factors. In case of convergence difficulties, time was dropped from the random effects. Fixed effects included time, group, time×group interaction terms and covariates as described. Categorical variables were effect-coded, time was centered. To assess main effects and interactions of variables with more than one coefficient (time, time×group interaction), Type II F-tests were conducted using Satterthwaite's degrees of freedom method.

If the assumptions of a statistical analysis were violated in the efficacy analyses despite efforts of transformation, alternative tests were used. If the assumptions for mixed models were violated, RM ANOVAs were used. If the assumptions for RM ANOVAs were violated, one-way ANOVAs were performed. If the assumptions for parametric testing were violated, non-parametric tests were calculated. Time effects were then evaluated by Wilcoxon Signed-Rank tests or Friedman ANOVAs, group effects by Mann Whitney U tests, and time×group interactions by Mann Whitney U tests on difference values between measurements (e.g. group difference in the difference value between HR sitting before the TSST minus HR standing before the TSST).

Subgroup analyses were performed for the different strata, i.e. female participants, male participants, high stressed participants and low stressed participants.

For all endpoints, analyses were performed for the Intent-To-Treat (ITT) and Per Protocol (PP) populations, separately. For the PP analyses, individual decisions on exclusion of participants or data points were made during the Blind Data Review.

### Determination of Sample Size:

The estimated sample size was computed for a repeated measurement ANOVA with:
2 groups
7 measurements
a small effect size of f=0.1
α-error probability=0.05.
power (1-β-error probability)=0.85
   and resulted in a group size of 56 participants each. In order to account for protocol deviations, the estimated sample size was rounded up to a total sample size of 60 participants per treatment group.

### Data Sets Analyzed.

### ITT Population

This population included all randomized participants who satisfied the inclusion/exclusion criteria. The ITT population contained 120 participants, with data available for all endpoints for 117 participants.

### PP Population

The PP population is defined twice: once overall and once per endpoint, as some protocol deviations only effect certain endpoints.

The overall PP included all randomized participants who satisfied the inclusion/exclusion criteria and had no protocol violations. It contained 113 participants. The PP population was identified prior to database lock after the Blind Data Review.

Participants who had any of the following major violations to the protocol were excluded from the PP population:
1. consumed <80% of investigational product
2. did not participate at clinic visits V2 and V3
3. participated in binge drinking during the intervention period
4. excessively consumed caffeine during the intervention period
5. consumed antibiotics during the intervention period
6. consumed psychoactives during the intervention period
7. significantly changed smoking habits during the intervention period
8. significantly changed diet during the intervention period

For individual endpoints, participants were excluded, if they showed deviations that were considered to possibly affect the endpoint in question. The number of participants analyzed in the PP thus was different for each endpoint.

### Example 1: HR in Response to the TSST

HR was expected to show an increase in response to the TSST and a subsequent decrease afterwards, irrespective of treatment group. Efficacy was defined as an attenuated increase in HR over the course of the TSST (from sitting pre-TSST to sitting post-TSST) for the active group compared to the placebo group. HR was averaged for the individual seven-time windows (see above Physiological Measures—the Autonomic Nervous System (ANS)-Heart Rate (HR)). Group differences were not assessed by comparing values for individual time windows, but by comparing the two trajectories of HR over time. Time was coded as a continuous variable. HR over time was modelled by a curved line by means of including higher order terms for time in addition to a linear effect, which captured an overall increase or decrease. Statistically, efficacy was operationalized as an interaction between treatment group and time. A significant interaction stated that the two-time curves for the two treatment groups differed.

### Example 2: VAS-Exhaustion in Response to the TSST

Irrespective of treatment group, VAS exhaustion was expected to increase in response to the TSST yielding higher values during the TSST than post-TSST, compared to pre-TSST. Efficacy was defined as a reduced increase from pre-TSST to during-TSST or to post-TSST for the active group as compared to the placebo group and operationalized as the interaction between time and treatment group. Time was coded as a continuous variable and was only included as a linear term.

### Example 3: Systolic Blood Pressure in Response to the TSST

Irrespective of treatment group, blood pressure was expected to increase in response to the TSST yielding higher values in the post-TSST measurements, compared to the pre-TSST measurements. Efficacy was defined as a reduced increase for the active group as compared to the placebo group and operationalized as the interaction between time and treatment group. Time was coded as a categorical variable with the two-time points pre-TSST and post-TSST.

### Example 4: Perceived Stress (PSS) in Response to Treatment—Baseline Vs End of Study

Irrespective of treatment group, stress perception was not necessarily expected to change between pre-treatment and post-treatment. Efficacy was defined as a decrease, or (in case of a general increase) reduced increase for the active group as compared to the placebo group and operationalized as the interaction between time and treatment group. Time was coded as a categorical variable with the two-time points pre-treatment and post-treatment.

### Example 5: 8 pm Cortisol in Response to Treatment—Baseline Vs End of Study

In addition to the CAR variables, the cortisol level at 8 μm was analyzed. These cortisol indices are frequently used to describe HPA-axis activity and represent information either of the total cortisol production or of the change in cortisol levels.

Efficacy for cortisol at 8 μm was defined in terms of a normalization: Number of participants with normal values (between first and third quantile of reference measures) and numbers of participants with low or high values are compared before treatment and after treatment. More participants in the normal range after treatment was defined as efficacy.

### Example 6: Diastolic Blood Pressure in Response to Treatment—Baseline Vs End of Study

Irrespective of treatment group, blood pressure was not expected to change significantly in response to the treatment. Efficacy was defined as a decrease (in case of a general increase) reduced increase for the active treatment group as compared to the placebo group and operationalized as the interaction between time and treatment group. Time was coded as a categorical variable with the two-time points pre-treatment and post-treatment.

### Example 7: Sleep-Related Recovery Throughout the Study Period (Week 1 to Week 7)

Sleep related recovery was rated by participants on an 11-point scale (0-10) and monitored throughout the run-in period (week 1 and 2) and the subsequent treatment phase (weeks 3-7). Irrespective of treatment group, sleep related recovery was not necessarily expected to change over time. Efficacy was defined as an increase, or (in case of a general decrease) reduced decrease for the active group as compared to the placebo group and operationalized as the interaction between time and treatment group. Time was coded as a continuous variable with one value for each day and participant. Polynomials of time were included to be able to model the trajectory as a curved line.

### Example 8: Perceived Health Throughout the Study Period (Week 1 to Week 7)

Health status was rated by participants on an 11-point scale (0-10) and monitored through the run-in period (week 1 and 2) and the subsequent treatment phase (weeks 3-7). Irrespective of treatment group perceived health status was not necessarily expected to change over time. Efficacy was defined as an increase, or (in case of a general decrease) reduced decrease for the active group as compared to the placebo group and operationalized as the interaction between time and treatment group. Time was coded as a continuous variable with one value for each day and participant. Polynomials of time were included to be able to model the trajectory as a curved line.

### Example 9: Perceived Productivity Throughout the Study Period (Week 1 to Week 7)

Productivity was rated by participants on an 11-point scale (0-10) and monitored through the run-in period (week 1 and 2) and the subsequent treatment phase (weeks 3-7). Irrespective of treatment group perceived productivity was not necessarily expected to change over time. Efficacy was defined as an increase, or (in case of a general decrease) reduced decrease for the active group as compared to the placebo group and operationalized as the interaction between time and treatment group. Time was coded as a continuous variable with one value for each day and participant. Polynomials of time were included to be able to model the trajectory as a curved line.

### Results and Comments

### Heart Rate in Response to TSST

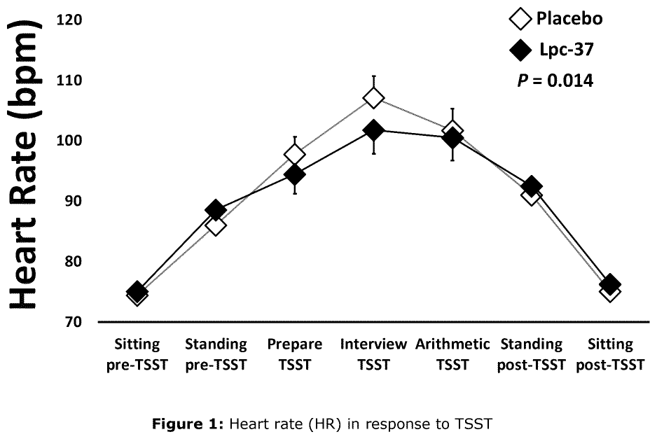

FIG. 1 shows the HR (bpm) in response to the TSST (mean values for individual time windows±SEM) following 5 weeks of daily intervention with 1.75×10¹⁰ CFU of Lacticaseibacillus paracasei Lpc-37 in the active group and compared with the placebo group. HR was continuously measured in individual participants from both groups using a Polar watch device worn by participants from pre-TSST (−20 min) to post-TSST (+20 min) at the end of the study (day 51).

Mean values were calculated for 7 different time windows throughout the TSST protocol at the end of the study (day 51). The time windows were as follows: sitting pre-TSST (−20 min), standing pre-TSST (−10 min), introduction to the TSST and preparation time (5 min), interview TSST (5 min), mental arithmetic task TSST (5 min), standing post-TSST (+10 min) and sitting post-TSST (+20 min). N=28 for the active group and N=28 for the placebo group and both groups had a TICS score ≤13 in the PP population.

Statistical analysis: the final model included four orthogonal polynomials for time, gender as a covariate, and treatment group and an interaction between treatment group and all-time components as fixed effects. Random slopes had to be dropped to avoid singular fits. HR was log-transformed to fulfill model assumptions. Time was centered. The interaction between treatment group and time was significant, indicating that the change in HR does depend on the treatment group (p=0.014). FIG. 1 shows that the increase in HR was smaller in the active group compared to placebo. Efficacy can be confirmed for the active treatment group.

bpm: beats per minute, CFU: Colony Forming Unit, HR: Heart Rate, PP: Per Protocol, TICS: Trier Inventory for Chronic Stress, TSST: Trier Social Stress Test, SEM: Standard Error of the Mean.

### VAS-Exhaustion in Response to TSST

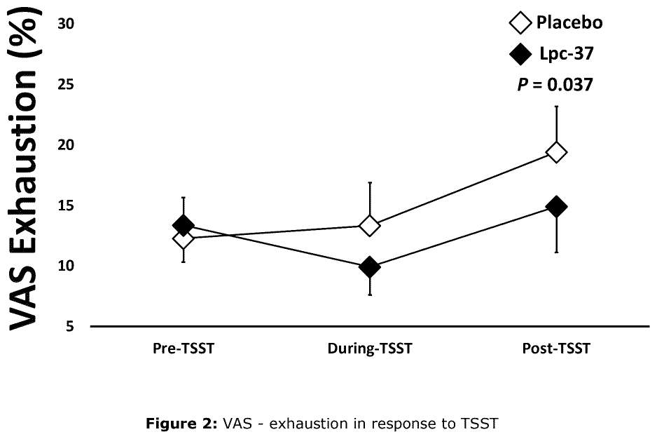

FIG. 2 shows VAS-exhaustion (%) in response to the TSST (mean values in individual time windows±SEM) following 5 weeks of daily intervention with 1.75×10¹⁰ CFU of Lacticaseibacillus paracasei Lpc-37 in the active group and compared with the placebo group. VAS-exhaustion was measured in both groups using a specific VAS to detect participants self-reported exhaustion levels pre-TSST (−10 min), during the TSST and post-TSST (+1 min) at the end of the study (day 51).

N=28 for the active group and N=29 for the placebo group and both groups had a TICS score ≤13 in the PP population.

Statistical analysis: The final linear mixed effects model included only a linear component of time, STAI-trait was a covariate and treatment group and an interaction between treatment group and time as fixed effects. The random slope for time had to be dropped to reach convergence in some of the subgroup analyses. The VAS percentage was square root-transformed to fulfill model assumptions. The interaction between treatment group and time was significant, indicating that the change in VAS-exhaustion ratings does depend on the treatment group (p=0.037). FIG. 2 shows that the increase in VAS exhaustion was smaller for the active group compared to placebo. Efficacy can be confirmed for the active treatment group.

CFU: Colony Forming Unit, PP: Per Protocol, SEM: Standard Error of the Mean, STAI: State Trait Anxiety Inventory, TICS: Trier Inventory for Chronic Stress, TSST: Trier Social Stress Test, VAS: Visual Analogue Scale.

### Systolic Blood Pressure in Response to TSST

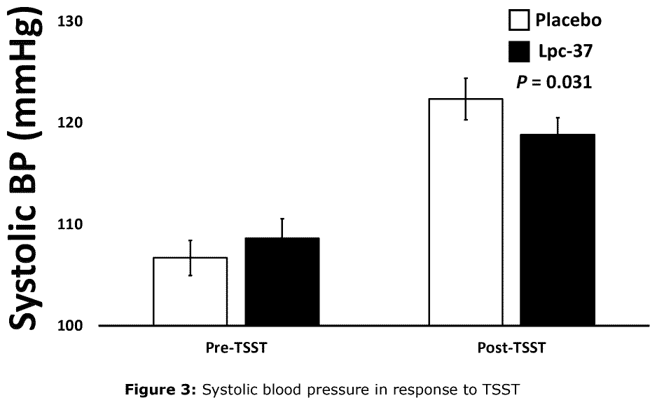

FIG. 3 shows systolic blood pressure (mmHg) in response to the TSST (mean values for individual time windows±SEM) following 5 weeks of daily intervention with 1.75×10¹⁰ CFU of Lacticaseibacillus paracasei Lpc-37 in the active group and compared with the placebo group. Systolic blood pressure was measured in both groups using a specific automated blood pressure measurement device at pre-TSST (−3 min) and post-TSST (+1 min) at the end of the study (day 51).

N=29 for the active group and N=28 for the placebo group in the PP population.

Statistical analysis: For systolic blood pressure, repeated measures ANOVAs were used. FIG. 3 shows that for systolic blood pressure, there was a significant treatment by time interaction (p=0.031). Systolic blood pressure increased less between the pre-TSST and the post-TSST measurement in the active group compared to the placebo group. Efficacy can be confirmed for the active treatment group.

CFU: Colony Forming Unit, mmHg: Millimeter of Mercury, PP: Per Protocol, SEM: Standard Error of the Mean, TSST: Trier Social Stress Test.

### Perceived Stress-Baseline Vs End of Study

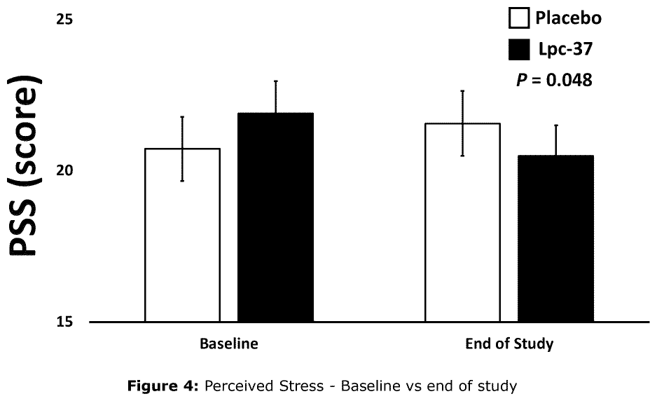

FIG. 4 shows the perceived stress (PSS score) in response to the treatment (mean values in individual time windows±SEM) following 5 weeks of daily intervention with 1.75×10¹⁰ CFU of Lacticaseibacillus paracasei Lpc-37 in the active group and compared with the placebo group. Perceived stress was measured in both groups using the PSS taken at baseline (day 16) and at the end of the study (day 51). N=55 for the active group and N=57 for the placebo group in the PP population.

Statistical analysis: Repeated measures ANOVAs including sex and chronic stress as covariates were calculated. The interaction between treatment group and time was significant (p=0.048), indicating that the change in perceived stress depends on the treatment group. Efficacy can be confirmed for the active treatment group.

ANOVA: Analysis of Variance, CFU: Colony Forming Unit, PP: Per Protocol, PSS: Perceived Stress Scale, SEM: Standard Error of the Mean.

### 8 pm Cortisol in Response to Treatment

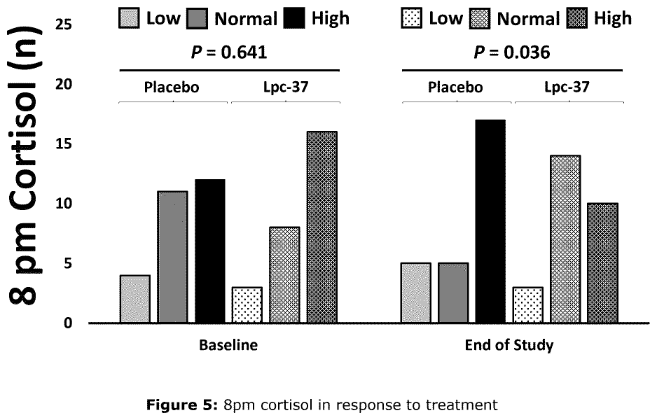

FIG. 5 shows cortisol normalization at 8 pm (cortisol frequencies; number of people with normal 8 pm cortisol response) in response to the treatment following 5 weeks of daily intervention with 1.75×10¹⁰ CFU of Lacticaseibacillus paracasei Lpc-37 in the active group and compared with the placebo group. Cortisol normalization at 8 μm was measured in both groups from saliva samples taken from individual participants in each group during days 14-15 (baseline) and during days 49-50 (end of study). The number of participants with normal values (between first and third quantile of reference measures) and numbers of participants with low (below first quantile of reference measures) or high (above third quantile of reference measures) values were compared before (days 14-15) and after (days 49-50) treatment.

N=27 for the active group and N=27 for the placebo group and both groups had a TICS score ≤13 in the PP population.

Statistical analysis: Fischer's exact tests were used to test for potential differences between treatment groups in the cortisol test value categories (for each variable: low, normal, and high cortisol values). RM ANOVAs and RM ANCOVAs were performed to estimate differences between treatment groups over time.

Here, 2 (before treatment, after treatment)×2 (active group, placebo group) ANOVAs were performed for the cortisol levels at 8 μm. The distribution of participants in different cortisol test value categories does not depend on the treatment group before treatment (p=0.641) but does depend on the treatment group after treatment (p=0.036). Efficacy on cortisol at 8 pm can be confirmed for the active treatment group

ANCOVA: Analysis of Covariance, ANOVA: Analysis of Variance, CFU: Colony Forming Unit, PP: Per Protocol, TICS: Trier Inventory for Chronic Stress.

### Diastolic Blood Pressure in Response to Treatment-Baseline Vs End of Study

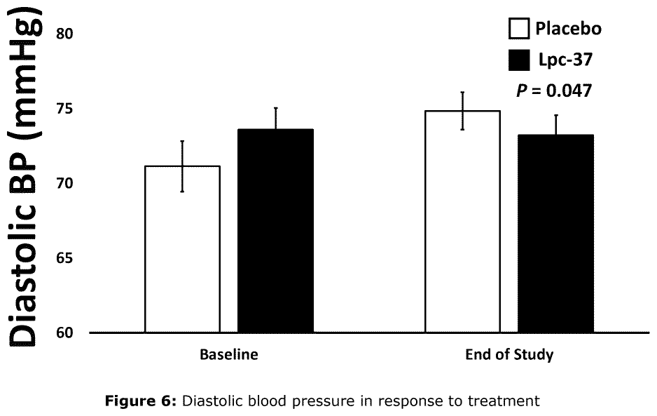

FIG. 6 shows diastolic blood pressure (mmHg) in response to treatment (mean values for individual time windows±SEM) following 5 weeks of daily intervention with 1.75×10¹⁰ CFU of Lacticaseibacillus paracasei Lpc-37 in the active group and compared with the placebo group. Diastolic blood pressure was measured in both groups using a specific automated blood pressure measurement device at baseline (V2; day 16) and end of study (V3; day 51).

N=27 for the active group and N=29 for the placebo group in the PP population and both groups had a TICS score ≥13 in the PP population.

Statistical analysis: For two participants, baseline measures were missing. For these participants, the post-treatment measurements were also dropped from the inferential analysis as the method employed was limited to complete case analysis but retained in the descriptive statistics. Repeated measures ANOVAS were used. FIG. 6 shows that for diastolic blood pressure, there was a significant treatment by time interaction (p=0.047). Diastolic blood pressure increased from pre-treatment to post-treatment in the placebo group while it stayed stable in the active group. Efficacy can be confirmed for the active treatment group.

ANOVA: Analysis of Variance, CFU: Colony Forming Unit, mmHg: Millimeter of Mercury, PP: Per Protocol, SEM: Standard Error of the Mean, TSST: Trier Social Stress Test.

### Sleep-Related Recovery Throughout Study Period

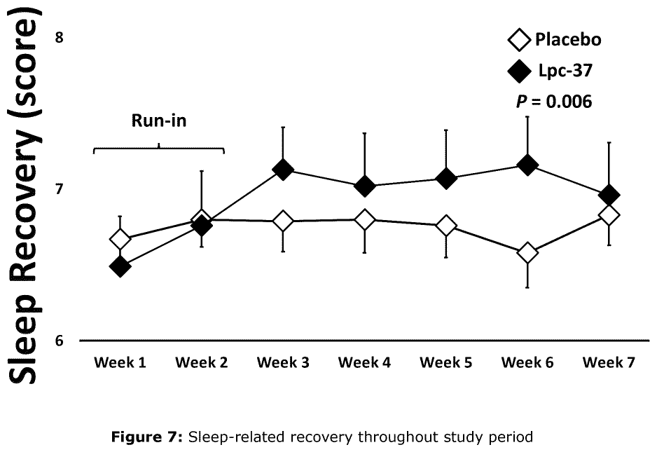

FIG. 7 shows sleep-related recovery throughout the study period (day 2-51) following 2 weeks of run-in period and 5 weeks of daily intervention with 1.75×10¹⁰ CFU of Lacticaseibacillus paracasei Lpc-37 in the active group and compared with the placebo group (mean values for individual time windows±SEM).

Sleep-related recovery was measured in both groups by averaging the individual responses from participants in each group for individual time windows (week) to the question “on a scale of 1 (not at all) to 10 (very), how rested do you feel today?”.

N=19 for the active group and N=25 for the placebo group and both groups had a TICS score ≥14 in the PP population.

Statistical analysis: The final model includes three orthogonal polynomials of time, gender and STAI trait scores as covariates and treatment group and an interaction between treatment group and all-time components as fixed effects. Recovery ratings were square-transformed to fulfill model_assumptions and the time variable was centered and scaled. There were three missing values in the PP and week means were calculated irrespective of missing values and participant records containing missing day values were retained in the analyses. The interaction between treatment group and time was significant, indicating that the change in sleep-related recovery is dependent on the treatment group (p=0.006). Efficacy can be confirmed for the active treatment group.

CFU: Colony Forming Unit, PP: Per Protocol, SEM: Standard Error of the Mean, STAI: State Trait Anxiety Inventory, TICS: Trier Inventory for Chronic Stress.

### Perceived Health Throughout Study Period

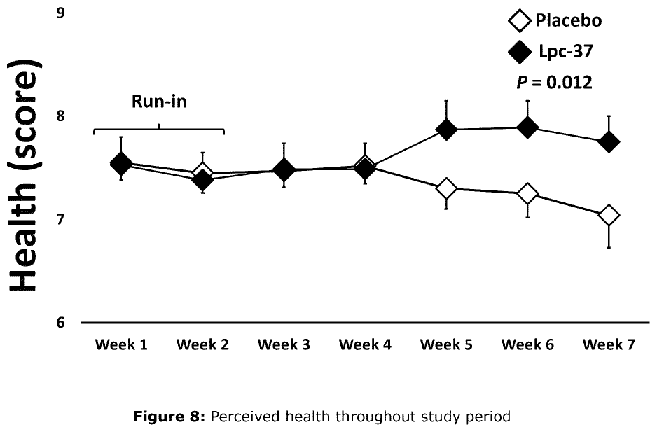

FIG. 8 shows the perceived health status throughout the study period (day 2-51) following 2 weeks of run-in period and 5 weeks of daily intervention with 1.75×10¹⁰ CFU of Lacticaseibacillus paracasei Lpc-37 in the active group and compared with the placebo group (mean values for individual time windows±SEM). Perceived health status was measured in both groups by averaging the individual responses from participants in each group for individual time windows (week) to the question “on a scale of 1 (not at all) to 10 (very), how healthy do you feel today?”.

N=19 for the active group and N=25 for the placebo group and both groups had a TICS score ≥14 in the PP population.

Statistical analysis: Model assumptions were violated for the linear mixed model; therefore, repeated measures ANOVAs were calculated after averaging the outcome by week. Perceived health ratings were square-transformed to fulfill model_assumptions. There were 10 missing values in the PP and week means were calculated irrespective of missing values and participant records that contained missing day values were retained in the analyses. The interaction between treatment group and time was significant (p=0.012). Efficacy can be confirmed for the active treatment group.

ANOVA: Analysis of Variance, CFU: Colony Forming Unit, PP: Per Protocol, SEM: Standard Error of the Mean, TICS: Trier Inventory for Chronic Stress.

### Perceived Productivity Throughout Study Period

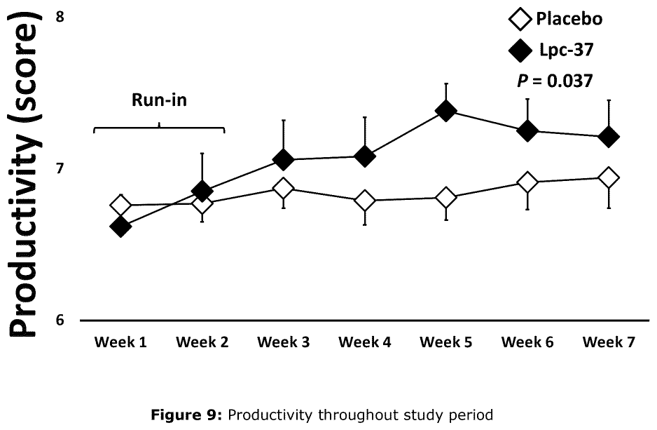

FIG. 9 shows the perceived productivity throughout the study period (day 2-51) following 2 weeks of run-in period and 5 weeks of daily intervention with 1.75×10¹⁰ CFU of Lacticaseibacillus paracasei Lpc-37 in the active group and compared with the placebo group (mean values for individual time windows±SEM). Perceived productivity was measured in both groups by averaging the individual responses from participants in each group for individual time windows (week) to the question “on a scale of 1 (not at all) to 10 (very), how productive do you feel today?”.

N=29 for the active group and N=30 for the placebo group and both groups had a TICS score ≥14 in the ITT population.

Statistical analysis: Model assumptions were violated for the linear mixed model on the raw data, therefore data were aggregated by calculating the week means for each participant. The linear mixed model includes three orthogonal polynomials of week, STAI trait scores as a covariate and treatment group and the interaction between treatment group and all-time components. There were 10 missing values in the ITT and week means were calculated irrespective of missing values and participant records containing missing day values were retained in the analyses. The interaction between treatment group and time was significant, indicating that the change in productivity ratings does depend on the treatment group (p=0.037). The increase in productivity was larger in the active group compared to the placebo group. Efficacy can therefore be confirmed for the active treatment group.

CFU: Colony Forming Unit, PP: Per Protocol, SEM: Standard Error of the Mean, STAI: State Trait Anxiety Inventory.

General Conclusions from the Results:

The following conclusions can be drawn from the results described above. Treatment with Lacticaseibacillus paracasei Lpc-37:
reduced the increase in HR in response to the TSST, compared to placebo. HR is a physiological response to psychosocial and/or psychological stress (FIG. 1).
reduced the increase of perceived exhaustion in response to the TSST, compared to placebo. Exhaustion is a psychological response to psychosocial and/or psychological stress (FIG. 2).
reduced the increase in systolic blood pressure in response to the TSST, compared to placebo. Systolic blood pressure is a physiological response to psychosocial and/or psychological stress (FIG. 3).
reduced the perception of stress, compared to placebo. Increased perceived stress is a symptom affecting mental health (FIG. 4).
promoted the normalization cortisol production as measured by the 8 pm cortisol production, compared to placebo. Increased cortisol production is a symptom affecting mental health (FIG. 5).
reduced the increase in diastolic blood pressure, compared to placebo. Diastolic blood pressure is a symptom affecting mental health (FIG. 6).
increased sleep related recovery (how rested you feel after a night sleep) over the course of the treatment, compared to placebo. Sleep disruptions and sleep related recovery are symptoms affecting mental health (FIG. 7).
increased the perception of health over the course of the treatment, compared to placebo. Mental and overall well-being is promoted by improvements to perceived health (FIG. 8).
increased the perception of productivity over the course of the treatment, compared to placebo. Mental and overall well-being is promoted by improvements to perceived productivity (FIG. 9).

1. Bacterium of the species Lacticaseibacillus paracasei for use in alleviating the psychological and/or physiological response to psychosocial and/or psychological stress and promoting mental and overall well-being, in a mammal.

2. Bacterium of the species Lacticaseibacillus paracasei for use in preventing and/or treating a symptom affecting mental health and promoting mental and overall well-being, in a mammal.

3. The bacterium for use according to claim 1, wherein the psychological response to psychosocial and/or psychological stress is perceived exhaustion.

4. The bacterium for use according to claim 1, wherein the physiological response to psychosocial and/or psychological stress is increased heart rate, and/or salivary cortisol production and/or blood pressure.

5. The bacterium for use according to claim 2, wherein the symptom affecting mental health is increased salivary cortisol production, and/or blood pressure, and/or perceived stress, and/or perceived anxiety, and/or sleep disruptions.

6. The bacterium for use according to claim 2, wherein the symptom affecting mental health may contribute to a neuropsychiatric condition and/or mental illness.

7. The bacterium for use according to claim 6, wherein the neuropsychiatric condition includes degenerative diseases, such as dementia, Parkinson's disease, Alzheimer's disease; mood disorders such as depression; neurotic disorders such as anxiety disorder and sleep disorders such as sleep apnea, narcolepsy, insomnia and parasomnia.

8. The bacterium for use according to claim 1 or 2, wherein the mental and overall well-being is promoted by improvements to perceived health and/or perceived productivity.

9. The bacterium for use according to claim 1 or 2, wherein the bacterium of the species Lacticaseibacillus paracasei is a probiotic of the species Lacticaseibacillus paracasei or a mixture thereof.

10. The bacterium for use according to any one of the claims 1-9, wherein the bacterium of the species Lacticaseibacillus paracasei is strain Lpc-37, registered at the DSMZ under deposit number DSM32661, on 5 Oct. 2017.

11. A composition comprising a bacterium of the species Lacticaseibacillus paracasei for use in alleviating the psychological and/or physiological response to psychosocial and/or psychological stress and promoting mental and overall well-being, in a mammal.

12. A composition comprising a bacterium of the species Lacticaseibacillus paracasei for use in preventing and/or treating a symptom affecting mental health and promoting mental and overall well-being, in a mammal.

13. The composition for use according to claim 11, wherein the psychological response to psychosocial and/or psychological stress is perceived exhaustion.

14. The composition for use according to claim 11, wherein the physiological response to psychosocial and/or psychological stress is increased heart rate, and/or salivary cortisol production and/or blood pressure.

15. The composition for use according to claim 12, wherein the symptom affecting mental health is increased salivary cortisol production, and/or blood pressure, and/or perceived stress, and/or perceived anxiety, and/or sleep disruptions.

16. The composition for use according to claim 12, wherein the symptom affecting mental health may contribute to a neuropsychiatric condition.

17. The composition for use according to claim 16, wherein the neuropsychiatric condition includes degenerative diseases, such as dementia, Parkinson's disease, Alzheimer's disease; mood disorders such as depression; neurotic disorders such as anxiety disorder and sleep disorders such as sleep apnea, narcolepsy, insomnia and parasomnia.

18. The composition for use according to claim 11 or 12, wherein the mental and overall well-being is promoted by improvements to perceived health and/or perceived productivity.

19. The composition for use according to claim 11 or 12, wherein the bacterium of the species Lacticaseibacillus paracasei is a probiotic of the species Lacticaseibacillus paracasei or a mixture thereof.

20. The composition according to any one of the claims 11-19, wherein the bacterium of the species Lacticaseibacillus paracasei is strain Lpc-37, registered at the DSMZ under deposit number DSM32661, on 5 Oct. 2017.

21. The composition according to any of claims 11-20, wherein the composition is in the form of a food product, a dietary supplement or a pharmaceutically acceptable composition.

22. The composition according to any of claims 11-20, wherein the composition is a spray-dried or freeze-dried composition.

23. The composition according to claim 22, wherein the composition comprises a cryoprotectant.

24. The composition according to any of the claims 11-23, wherein the bacterium of the species Lacticaseibacillus paracasei is present in the composition in an amount between 10⁶ and 10¹², e.g. between 10⁸ and 10¹² CFU per dose, optionally 10¹⁰ CFU per dose.
